(19) United States
(12) Patent Application Publication
(10) Pub. No.: US 2025/0366798 A1
(43) Pub. Date: Dec. 4, 2025

(54) TARGET VOLUME TRACKING METHOD AND COMPUTER DEVICE

(71) Applicant: Shanghai United Imaging Healthcare Co., Ltd., Shanghai (CN)

(72) Inventors: Xiangbin Fang, Shanghai (CN); Can Liao, Shanghai (CN)

(21) Appl. No.: 19/221,507

(22) Filed: May 29, 2025

(30) Foreign Application Priority Data
May 29, 2024 (CN) 202410684126.9

(51) Int. Cl. A61B 6/03; A61B 6/00; A61B 6/40; A61B 6/42

(52) U.S. Cl. CPC A61B 6/03; A61B 6/4064; A61B 6/4291; A61B 6/5264

(57) ABSTRACT

The present disclosure relates to a target volume tracking method and a computer device. The method includes: determining an isocenter location corresponding to the target volume in a scout image of an object, and obtaining the spatial position of the treatment head in the radiotherapy device, and then determining the at least one slice on the scout image based on the isocenter location and the spatial position, so as to control the magnetic resonance scanner to radiate a RF pulse which excites the at least one slice to obtain a tracking image of the object; the tracking image includes the target volume.

### CROSS-REFERENCE TO RELATED APPLICATIONS

The present application claims priority to Chinese patent application No. 2024106841269, filed on May 29, 2024, and entitled “TARGET VOLUME TRACKING METHOD, APPARATUS AND COMPUTER DEVICE”, the entire content of which is incorporated herein by reference.

### TECHNICAL FIELD

The present disclosure relates to the field of radiotherapy technologies, and in particular, to a target volume tracking method and computer device.

### BACKGROUND

With the continuous advancement of radiotherapy technologies, dynamic radiotherapy technologies are becoming key development directions of radiotherapy technologies. In dynamic radiotherapy technologies, the target volume is tracked by a medical imaging device, and movement of a grating of a treatment head is controlled based on the tracked target volume, so that the treatment head can follow the target volume for more accurate radiotherapy.

However, the accuracy of target volume tracking in the dynamic radiotherapy technologies is not high at present. Therefore, how to propose a target volume tracking method with higher accuracy is the focus of research by those skilled in the field.

### SUMMARY

A target volume tracking method and computer device are provided in the present disclosure.

In a first aspect, a target volume tracking method is provided in the present disclosure. The target volume tracking method is applied to a magnetic resonance guided radiotherapy system. The magnetic resonance guided radiotherapy system includes a magnetic resonance scanner and a radiotherapy device. The method includes determining an isocenter location corresponding to the target volume in a scout image of an object, obtaining a spatial position of a treatment head in the radiotherapy device, determining at least one slice to be excited on the scout image based on the isocenter location and the spatial position, and controlling the magnetic resonance scanner to radiate a RF pulse which excites the at least one slice to obtain a tracking image of the object. The tracking image includes the target volume.

In an embodiment, determining the isocenter location corresponding to the target volume in the scout image of the object includes obtaining the scout image by the magnetic resonance scanner, and determining the isocenter location in the scout image.

In some embodiments, determining the isocenter location in the scout image includes: determining the isocenter location based on the scout image and a coordinate transformation relationship. The coordinate transformation relationship includes a transformation relationship between a real space coordinate system and an image coordinate system corresponding to the scout image.

In some embodiments, the at least one slice of the object is affected by respiratory movement or cardiac movement.

In some embodiments, the at least one slice is a two-dimensional slice.

In some embodiments, the angle between the at least one slice and a beam direction of the treatment head is greater than or equal to 80°, and less than or equal to 110°.

In some embodiments, an angle between the at least one slice and the beam direction of the treatment head is 90°.

In some embodiments, the spatial position includes real-time angular information of the treatment head attached to a rotatable gantry of the radiotherapy device. Determining the at least one slice on the scout image, based on the isocenter location and the spatial position, includes determining a scanning angle of the at least one slice on the scout image based on the real-time angular information, and identifying a median axis of the target volume based on the isocenter location.

In some embodiments, determining the at least one slice on the scout image based on the isocenter location and the spatial position, includes determining a line connecting the isocenter location and the spatial position, determining a circular section where the line and a gantry of the treatment head are located, and determining, based on the isocenter location, the at least one slice parallel to the circular section on the scout image.

In some embodiments, the method further includes controlling the movement of a grating of the treatment head based on a planning image of the target volume and the tracking image.

In some embodiments, controlling the movement of a grating of the treatment head based on the planning image of the target volume and the tracking image, includes determining a first target volume in the planning image and a second target volume in the tracking image, determining an offset between the first target volume and the second target volume, and controlling the movement of the grating of the treatment head based on the offset.

In a second aspect, a target volume tracking method is further provided in the present disclosure. The method is applied to a magnetic resonance guided radiotherapy system. The magnetic resonance guided radiotherapy system includes a magnetic resonance scanner and a radiotherapy device. The method includes obtaining a scout image of an object by the magnetic resonance scanner, obtaining a spatial position of a treatment head in the radiotherapy device, determining an imaging angle of the magnetic resonance scanner based on the scout image of the object and the spatial position, controlling, based on the imaging angle of the magnetic resonance scanner, the magnetic resonance scanner to scan the object to obtain a tracking image, and controlling, based on a planning image of the target volume and the tracking image, the radiotherapy device to adjust the movement of a grating of the treatment head or adjust a shape of a beam generated by the radiotherapy device. The tracking image includes a target volume.

In a third aspect, a computer device is further provided in the present disclosure. The computer device includes a memory and a processor. The memory stores a computer program. The processor, when executing the computer program, implements determining an isocenter location corresponding to a target volume in a scout image of an object, obtaining a spatial position of a treatment head in a radiotherapy device, determining at least one slice to be excited on the scout image based on the isocenter location and the spatial position, and generating an imaging instruction based on the at least one slice. The imaging instruction is configured to control a medical imaging device to radiate a RF pulse which excites the at least one slice to obtain a tracking image of the object. The tracking image includes the target volume.

In some embodiments, the computer program, when executed by the processor, causes the processor to further implement: obtaining the scout image by the medical imaging device, and determining the isocenter location in the scout image.

In some embodiments, the computer program, when executed by the processor, causes the processor to further implement determining the isocenter location based on the scout image and a coordinate transformation relationship. The coordinate transformation relationship includes a transformation relationship between a real space coordinate system and an image coordinate system corresponding to the scout image.

In some embodiments, the computer program, when executed by the processor, causes the processor to further implement generating a control instruction based on a planning image of the target volume and the tracking image. The control instruction is configured to control movement of a grating of the treatment head.

In some embodiments, the computer program, when executed by the processor, causes the processor to further implement generating a control instruction based on a planning image of the target volume and the tracking image. The control instruction is configured to control a shape of a beam generated by the radiotherapy device.

In some embodiments, the computer program, when executed by the processor, causes the processor to further implement: determining a scanning angle of the at least one slice on the scout image based on real-time angular information of the treatment head attached to a rotatable gantry of the radiotherapy device, and determining a center position of the at least one slice on the scout image based on the isocenter location. The spatial position includes the real-time angular information.

In some embodiments, the computer program, when executed by the processor, causes the processor to further implement: determining a line connecting the isocenter location and the spatial position, determining a circular section where the line and a gantry of the treatment head are located, and determining the at least one slice parallel to the circular section on the scout image, based on the isocenter location.

In some embodiments, the computer program, when executed by the processor, causes the processor to further implement controlling the movement of a grating of the treatment head based on a planning image of the target volume and the tracking image.

### BRIEF DESCRIPTION OF THE DRAWINGS

In order to describe the technical solutions in the embodiments of the present application or the related technology more clearly, the following will briefly introduce the accompanying drawings required for describing the embodiments or the conventional technology. Apparently, the accompanying drawings in the following description are merely embodiments of the present disclosure, and for a person of ordinary skill in the art, other drawings can be obtained based on the disclosed drawings without creative efforts.

FIG. 1 is a schematic diagram illustrating stitching distortion in a tracking image.

FIG. 2 is a schematic diagram illustrating an application environment of a target volume tracking method in an embodiment of the present disclosure.

FIG. 3 is a flow diagram illustrating a target volume tracking method in an embodiment of the present disclosure.

FIG. 4 is a flow diagram illustrating a process of determining at least one slice to be excited in an embodiment of the present disclosure.

FIG. 5 is a flow diagram illustrating a process of determining at least one slice to be excited in another embodiment of the present disclosure.

FIG. 6 is a schematic diagram illustrating a process of determining at least one slice to be excited in an embodiment of the present disclosure.

FIG. 7 is a flow diagram illustrating a process of controlling movement of a grating in an embodiment of the present disclosure.

FIG. 8 is a schematic diagram illustrating a target volume tracking method in an embodiment of the present disclosure.

FIG. 9 is a schematic diagram illustrating a target volume tracking method in an embodiment of the present disclosure.

FIG. 10 is a flow diagram illustrating a target volume tracking method in another embodiment of the present disclosure.

FIG. 11 is a schematic diagram illustrating the effect of target volume tracking in an embodiment of the present disclosure.

FIG. 12 is a flow diagram illustrating a target volume tracking method in another embodiment of the present disclosure.

FIG. 13 is a block diagram illustrating a configuration of a target volume tracking apparatus in an embodiment of the present disclosure.

FIG. 14 is a block diagram illustrating a configuration of a target volume tracking apparatus in another embodiment of the present disclosure.

FIG. 15 is a diagram illustrating an internal configuration of a computer device in an embodiment of the present disclosure.

### DETAILED DESCRIPTION OF THE EMBODIMENTS

The technical solutions in the embodiments of the present disclosure will be clearly and completely described below with reference to the drawings in the embodiments of the present disclosure. Apparently, the described embodiments are only some but not all of the embodiments of the present disclosure. Based on the embodiments of the present disclosure, all other embodiments obtained by a person of ordinary skill in the art without creative efforts shall fall within the scope of protection of the present disclosure.

With the continuous advancement of radiotherapy technologies, it has developed from conventional square field radiotherapy technology to more advanced three-dimensional conformal radiotherapy technology, intensity-modulated radiotherapy technology, image-guided radiotherapy and adaptive radiotherapy technology. Three-dimensional conformal radiotherapy technology is a radiotherapy technology that obtains three-dimensional anatomical information of the patient's body parts through three-dimensional imaging technology and formulates a radiotherapy plan based on the three-dimensional anatomical information. Intensity-modulated radiotherapy is a radiotherapy technology that distributes the dose slice by slice by controlling the shape and intensity of the radiotherapy beam. Image-guided radiotherapy is a radiotherapy technology that uses imaging device to guide the radiotherapy before and during treatment. Adaptive radiotherapy is a radiotherapy technology that readjusts the radiotherapy plan in response to anatomical changes observed in the target volume between successive fractionated treatments.

In the future, dynamic radiotherapy technology that tracks target changes in real time will increasingly become a key development direction for high-precision radiotherapy. Magnetic resonance (MR) imaging is becoming an increasingly key imaging aid due to its excellent imaging capabilities for soft tissues and extremely high imaging freedom.

At present, the related MR imaging technologies include the following: (1) two-dimensional (2D) same-slice high temporal resolution imaging technology, (2) 2D orthogonal plane alternating real-time imaging technology, (3) multi-slice stitching imaging technology, and (4) three-dimensional (3D) volume real-time imaging technology.

2D same-slice high temporal resolution imaging technology may repeatedly acquire data on the same slice to ensure real-time update of the target volume position within the same slice. However, in this imaging technology, it is difficult to obtain three-dimensional motion information due to the singleness of the two-dimensional slice, i.e., the obtained tracking image has only two dimensions and lacks information of the third dimension.

2D orthogonal plane alternating real-time imaging technology may perform alternating acquisition based on the sagittal, coronal and transverse directions to locate the target volume in the three-dimensional direction. However, on the one hand, alternating imaging may cause cross artifacts in the tracking image, and there may be imaging delays in different plane directions. In the actual process of acquiring three-dimensional information, there is a time delay problem between the latter motion dimension information and the first two dimensions. On the other hand, the refresh rate of a single slice is fixed. Assuming that originally takes 0.2 seconds to obtain the tracking image of a certain slice, it takes 0.6 seconds to obtain the tracking image of the same slice after adopting the 2D orthogonal plane alternating real-time imaging technology. Therefore, the tracking image in the same direction may have the problem of low temporal resolution.

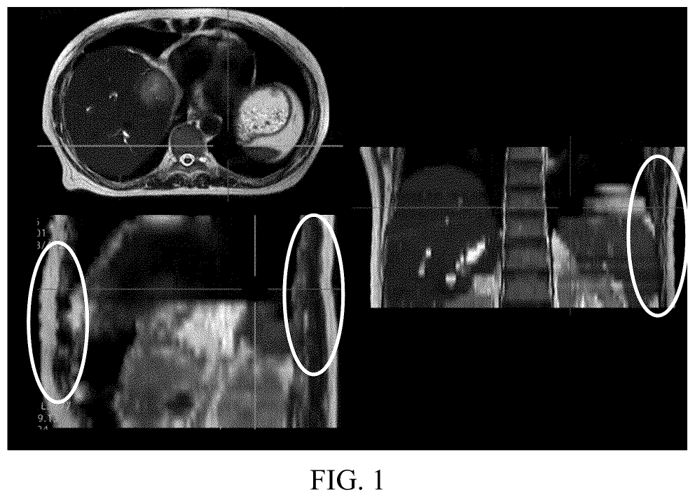

In the multi-slice stitching imaging technology, the acquisition direction remains unchanged, tracking images at different levels are acquired alternately and cyclically, and the tracking images at different levels are stitched together. However, due to machine limitations, the medical imaging device cannot accurately position each slice. Therefore, some geometric distortions may occur between different slices, resulting in stitching distortions in the stitched images. FIG. 1 is a schematic diagram illustrating stitching distortion in a tracking image. The stitching distortion can be implemented with reference to a white elliptical area in FIG. 1.

3D volumetric real-time imaging technology can improve the positioning accuracy of three-dimensional target volumes without inter-slice spacing problems, and the image coverage is the most comprehensive. However, under the premise of a certain coding efficiency, since adding one dimension of information requires collecting more two-dimensional slices, it is time-consuming. Therefore, this imaging technology has a high number of imaging coding steps, is difficult to improve the imaging speed, and puts great pressure on the stable operation of magnetic resonance imaging device. Moreover, this imaging technology is limited by the imaging time resolution and requires sacrificing a certain amount of image quality.

In view of the aforementioned technical problems, it is necessary to provide a target volume tracking method, so as to improve image quality and reduce the impact of image geometric deviation on the basis of achieving a higher imaging speed for target volume tracking. The target volume tracking method will be described below.

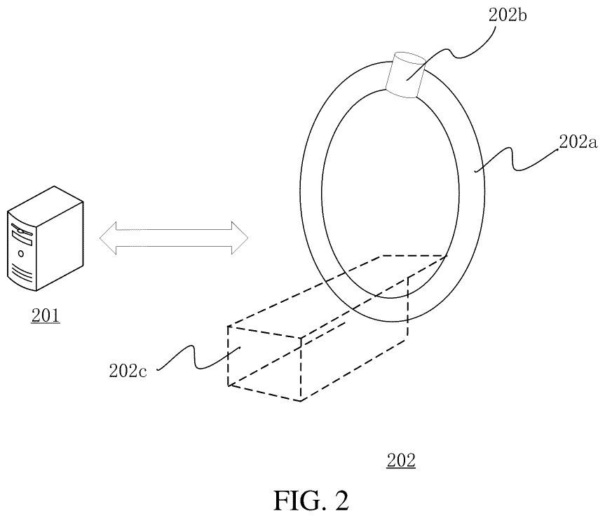

FIG. 2 is a schematic diagram illustrating an application environment of a target volume tracking method in an embodiment. The target volume tracking method provided in the embodiment of the present disclosure can be applied in the application environment shown in FIG. 2. A computer device 201 is capable of communicating with a radiation therapy system 202. The radiation therapy system 202 includes a medical imaging device and a radiotherapy device, the radiotherapy device can be integrated with the medical imaging device. The medical imaging device includes, but is not limited to, a magnetic resonance imaging (MRI) device, a computed tomography (CT) device, an ultrasound device, a positron emission computed tomography (PET)-CT device, and a PET-MR device.

The radiotherapy device may include a gantry 202a, a treatment head 202b on the gantry 202a, and a treatment couch 202c. The object can lie in a fixed area of the treatment couch 202c, so that the treatment couch can carry the object into an aperture of the gantry during movement, and then the gantry rotates to drive the treatment head to rotate. During the rotation process, a beam is projected to the target volume for radiotherapy.

The computer device 201 may be an independent physical server, or a server cluster or distributed system composed of multiple physical servers, or a cloud server providing cloud computing services. In some embodiments, the computer device 201 may also include but is not limited to various personal computers, laptops, smart phones, tablet computers, etc.

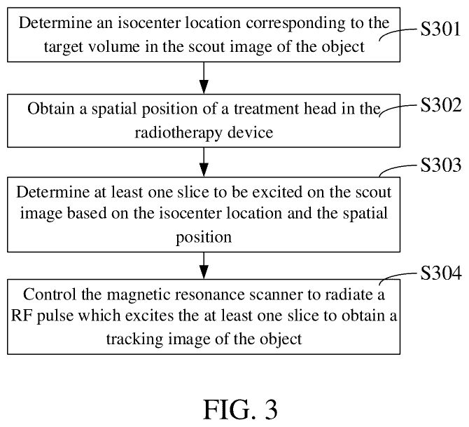

FIG. 3 is a flowchart illustrating a target volume tracking method in an embodiment. In an exemplary embodiment, as shown in FIG. 3, a target volume tracking method is provided. Taking an example where the method is applied to the computer device in FIG. 2, the radiation therapy system is a magnetic resonance guided radiotherapy system, and the medical imaging device is a magnetic resonance scanner. The method includes the steps S301 to S304.

In step S301, an isocenter location corresponding to the target volume in the scout image of the object is determined.

In this embodiment, the target volume is a region of the object that requires radiotherapy, which may include but is not limited to a tumor region. The MRI scanner first scans the object to obtain a scout image of the object. It can be understood that the scout image of the object includes the target volume, and further, the computer device can determine the isocenter location corresponding to the target volume in the scout image.

The isocenter location corresponding to the target volume may be the position information of the centroid of the target volume in the image coordinate system where the scout image is located, or may be the position information of the centroid of the target volume in the real space coordinate system. Optionally, the computer device may calculate the isocenter location corresponding to the target volume based on the scout image by a predetermined algorithm, or directly obtain the isocenter location corresponding to the target volume in the scout image of the object. For example, the computer device may also receive the isocenter location input by a user. In some embodiments, the computer device can further determine the isocenter location corresponding to the target volume based on the scout image and a coordinate transformation relationship. The coordinate transformation relationship can be a transformation relationship between a real space coordinate system and an image coordinate system corresponding to the scout image, which is not limited in this embodiment.

In step S302, a spatial position of a treatment head in the radiotherapy device is obtained.

In this embodiment, the spatial position of the treatment head in the radiotherapy device can represent the position information of the treatment head in the real space coordinate system. Exemplarily, the computer device may acquire the spatial position of the treatment head by a position sensor, such as an encoder.

In step S303, at least one slice to be excited on the scout image is determined based on the isocenter location and the spatial position.

In this embodiment, the at least one slice to be excited refers to an anatomical imaging slice to which the spatial coordinates (e.g., a scanning angle and a center position) of the at least one slice have been pre-selected and determined by a gradient magnetic field. The at least one slice requires medical imaging device (e.g., magnetic resonance scanner) to radiate a RF pulse for scan. Optionally, the at least one slice may be a two-dimensional slice.

The at least one slice can be determined by its plane equation, or by its the scanning angle and the center position, which is not limited in this embodiment.

It should be noted that since the position of the treatment head may change during radiotherapy, the position of the at least one slice may also change accordingly. Further optionally, the computer device may periodically update the spatial position to periodically determine the at least one slice based on the isocenter location and the spatial position.

Due to the presence of respiratory movement or cardiac movement of the object during radiotherapy, in an exemplary embodiment, optionally, the at least one slice of the object is affected by the respiratory movement or cardiac movement.

Furthermore, due to clinical application requirements, actual conformal therapy often requires the projection area of the target volume. Therefore, in an exemplary embodiment, optionally, the at least one slice can be determined based on the projection surface of the beam in the treatment head. Further optionally, an angle between the at least one slice and the beam direction of the treatment head is a predetermined value, and the predetermined value may be a value close to 90°, for example, any angle in a range from 80° to 110°. Exemplarily, the computer device may determine the projection surface of the beam in the treatment head based on the spatial position of the treatment head, and use the plane position passing through the isocenter location and parallel to the projection surface as the at least one slice.

Furthermore, during radiotherapy, only the respiratory movement or cardiac movement perpendicular to the beam is meaningful. Movement of the target volume in other directions cannot be covered by the beam. For example, when the beam is vertical from top to bottom, up and down movements of the target volume may not affect the projection area of the target volume in the up and down directions, but left and right movements of the target volume may affect the projection area of the target volume in the up and down directions. Based on this, it is sufficient to track the tracking image of the target volume in the vertical direction of the beam. Therefore, an angle between the at least one slice and the beam direction of the treatment head can be 90°. In this way, the optimal at least one slice in the current perpendicular beam direction can be adaptively scanned to extract the optimal trajectory for the present beam tracking.

In step S304, the magnetic resonance scanner is controlled to radiate a RF pulse which excites the at least one slice to obtain a tracking image of the object. The tracking image includes the target volume.

Furthermore, after the at least one slice is determined, the magnetic resonance scanner can be controlled to radiate a RF pulse which excites the at least one slice to obtain the tracking image including the target volume. In other words, the computer device can control the magnetic resonance scanner to scan the at least one slice to obtain a tracking image. Optionally, the computer device can control the magnetic resonance scanner to scan the at least one slice by controlling an excitation angle of a radio frequency pulse and an emission timing of a gradient pulse. The tracking image may include at least one two-dimensional image.

Exemplarily, the computer device may send an instruction including the at least one slice to the magnetic resonance scanner. After receiving the instruction, the magnetic resonance scanner may switch to the at least one slice of the object for scanning, and then a tracking image is obtained by the scanning.

It is understandable that at different slices, the computer device can obtain tracking images. Exemplarily, when the treatment head moves to a spatial position A, the computer device determines a slice A to be excited, and scans based on the slice A to obtain a tracking image A of the target volume. When the treatment head moves to a spatial position B, the computer device determines a slice B to be excited, and scans based on the slice B to obtain a tracking image B of the target volume, and so on. It should be noted that the above letters are only for distinction and do not limit the number of tracking images. In this way, the target volume can be tracked periodically during radiotherapy. In some embodiments, because the at least one slice is a two-dimensional slice, a tracking efficiency is higher than that of real-time imaging technology in a 3D volume.

Furthermore, tracking images can be used to define a radiation field. For example, after obtaining the tracking image A corresponding to the spatial position A, the radiation field can be adaptively adjusted according to the tracking image A to reduce the effects of respiratory movement, thereby improving the accuracy of radiotherapy.

In the above target volume tracking method, since the isocenter location corresponding to the target volume in the scout image of the object can be obtained, and the spatial position of the treatment head in the radiotherapy device can be obtained, the at least one slice can be determined on the scout image based on the isocenter location and the spatial position. In this way, the at least one slice is not fixed, nor does it change alternately only in three directions. Instead, it can be determined based on the spatial position of the treatment head in the radiotherapy device. Therefore, the at least one slice can be adaptively changed based on the beam of the treatment head. Furthermore, based on the determined at least one slice, the magnetic resonance scanner is controlled to scan the object to obtain the tracking image, so that a more accurate tracking image including the target volume can be obtained, thereby improving the accuracy of target volume tracking.

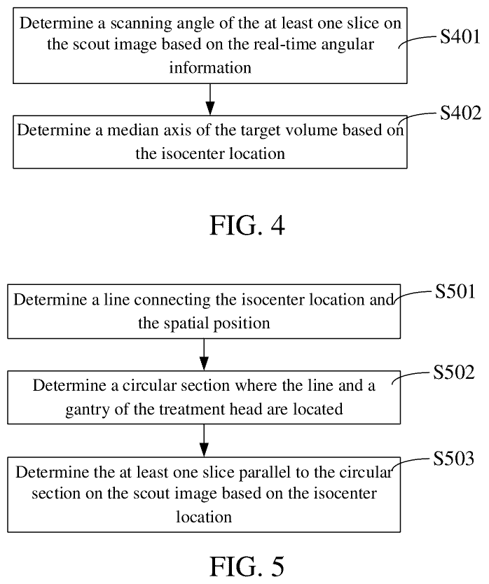

FIG. 4 is a flow diagram illustrating a process of determining at least one slice to be excited in an embodiment. In an exemplary embodiment, as shown in FIG. 4, the spatial position includes the real-time angular information of the treatment head attached to a rotatable gantry of the radiotherapy device. The at least one slice can be determined by the scanning angle and the center position on the scout image. Step S302 includes steps S401 to S402.

In step S401, a scanning angle of the at least one slice on the scout image is determined based on the real-time angular information.

In this embodiment, the spatial position includes real-time angular information of the treatment head attached to a rotatable gantry of the radiotherapy device. An isocenter of the radiotherapy device can be understood as a rotation center of the gantry, and the treatment head rotates based on the gantry. The real-time angular information of the treatment head can be configured to indicate a present rotation angle of the treatment head relative to the gantry. The rotation angle may be an angle between a first line and a second line. The first line is a line connecting the isocenter and the treatment head. The second line is a horizontal line passing through the isocenter. Exemplarily, taking a rotation of 360° as an example, in the initial state, the real-time angular information of the treatment head may be 0°, and after rotating ⅛ clockwise, the real-time angular information of the treatment head may be 45°.

Furthermore, the computer device can determine the scanning angle (FOV) of the at least one slice on the scout image based on the real-time angular information. Optionally, the computer device may obtain a scanning angle of the at least one slice after post-processing the real-time angular information, or the computer device may use the real-time angular information as the scanning angle of the at least one slice.

For example, assuming that the real-time angular information of the treatment head is 45°, the computer device can determine that the scanning angle of the at least one slice is also 45°.

In step S402, a median axis of the target volume is determined based on the isocenter location.

Furthermore, the computer device may determine the median axis of the target volume of the at least one slice on the scout image based on the isocenter location corresponding to the target volume. Optionally, the computer device may post-process the isocenter location to obtain the median axis of the target volume of the at least one slice.

The at least one slice can be uniquely determined based on the scanning angle and the center position. In other words, the spatial position of the treatment head can be used to determine the scanning angle, and the isocenter location corresponding to the target volume can be used to determine a rotation point of the at least one slice where the target volume is switched, i.e., the center position, to ensure that the target volume is always within a radiotherapy range during the rotation of the treatment head. Furthermore, the computer device can subsequently rotate the corresponding scanning angle relative to the center position of the at least one slice, thereby scanning the at least one slice to obtain a tracking image.

In the aforementioned embodiment, since the scanning angle of the at least one slice on the scout image can be determined based on the real-time angular information, and the center position of the at least one slice on the scout image can be determined based on the isocenter location. Therefore, the at least one slice is determined based on the scanning angle and the center position, i.e., a more accurate at least one slice can be efficiently determined based on the isocenter location and the spatial position.

FIG. 5 is a schematic diagram illustrating determining at least one slice to be excited in another embodiment. In an exemplary embodiment, as shown in FIG. 5, step S302 includes steps S501 to S503.

In step S501, a line connecting the isocenter location and the spatial position is determined.

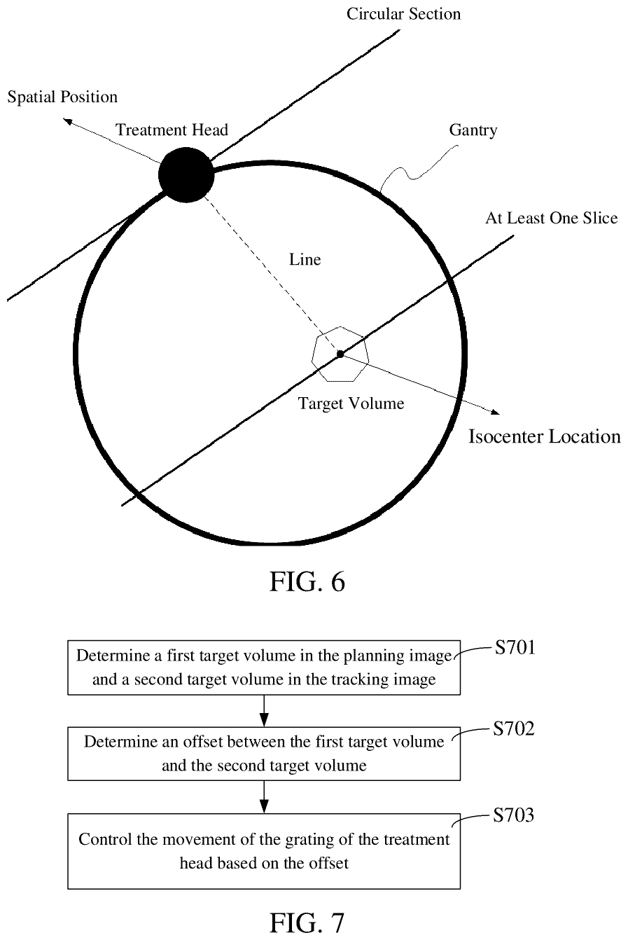

The method of determining the at least one slice will be described more clearly below with reference to FIG. 6. FIG. 6 is a schematic diagram illustrating a process of determining at least one slice to be excited in an embodiment. After the computer device determines the spatial position of the treatment head and the isocenter location corresponding to the target volume, the line connecting the isocenter location and the spatial position can be determined, as shown by the dotted line in FIG. 6.

In step S502, a circular section where the line and a gantry of the treatment head are located is determined.

Referring back to FIG. 6, the computer device can determine the circular section where the line and the gantry of the treatment head are located. In other words, the above line is a normal vector of the circular section.

In step S503, the at least one slice parallel to the circular section on the scout image is determined based on the isocenter location.

Furthermore, the computer device can determine the at least one slice parallel to the circular section on the scout image based on the isocenter location. Exemplarily, the computer device may use the plane position on the scout image that passes through the isocenter location and is parallel to the circular section as the at least one slice.

In the aforementioned embodiment, since the line connecting the isocenter location and the spatial position can be determined, and the circular section where the line and the gantry of the treatment head are located can be determined, the at least one slice on the scout image that is parallel to the circular section can be determined based on the isocenter location, thereby determining a more accurate at least one slice based on the isocenter location and the spatial position.

In an exemplary embodiment, optionally, the target volume tracking method further includes the following steps.

The movement of a grating of the treatment head is controlled based on a planning image of the target volume and the tracking image.

In this embodiment, the computer device can obtain the planning image of the target volume. For example, the medical imaging device may scan the target volume before surgery and send the planning image obtained after the scan to the computer device.

Furthermore, the computer device can control the movement of the grating of the treatment head based on the planning image and the tracking image. The grating of the treatment head may include but is not limited to a multi-leaf collimator system (MLC).

Continuing the above example, when the treatment head moves to a spatial position A, the computer device can control the movement of the grating of the treatment head based on the planning image and the tracking image A. When the treatment head moves to a spatial position B, the computer device can control the movement of a grating of the treatment head based on the planning image and the tracking image B, and so on.

Optionally, the computer device inputs the planning image and the tracking image into a trained model so that the model outputs motion parameters of the grating of the treatment head, and then the computer device controls the movement of the grating based on the motion parameters to perform dynamic radiotherapy. Exemplarily, the computer device can input the planning image and tracking image A into the model, and control the movement of a grating based on a motion parameter A output by the model, so that the treatment head can perform conformal radiotherapy more accurately when it is at the spatial position A.

In the aforementioned embodiments, since the movement of the grating of the treatment head is controlled based on the planning image of the target volume and the tracking image, the accuracy of the movement of the grating can be improved, thereby determining an accurate radiation field during the radiotherapy.

FIG. 7 is a schematic diagram illustrating a process of controlling of movement of a grating in an embodiment. In an exemplary embodiment, as shown in FIG. 7, the above-mentioned controlling the movement of the grating of the treatment head based on the planning image of the target volume and the tracking image includes steps S701 to S703.

In step S701, a first target volume in the planning image and a second target volume in the tracking image are determined.

In this embodiment, the computer device identifies the target volume in the planning image as the first target volume, and identifies the target volume in the tracking image as the second target volume, the computer device can determine the first target volume and the second target volume respectively through a threshold segmentation algorithm, and can determine the first target volume and the second target volume respectively through a trained recognition model. The computer device can further determine the first target volume and the second target volume respectively in response to the user's outlining operation, and this embodiment is not limited to this. Optionally, the ways of determining the first target volume and the second target volume may be different. For example, the first target volume in the planning image may be determined by a threshold segmentation algorithm, and the second target volume in the tracking image may be determined by a recognition model.

In step S702, an offset between the first target volume and the second target volume is determined.

Furthermore, the first target volume in the planning image can be used as a reference, and the offset between the first target volume and the second target volume can be determined based on the first target volume and the second target volume. The offset includes an offset size and an offset direction. For example, the computer device may determine that the second target volume is offset to the right by 1 centimeter (cm) relative to the first target volume.

In step S703, the movement of the grating of the treatment head is controlled based on the offset.

In this embodiment, the offset can indicate a movement direction of the target volume, and then the computer device can control a target volume of the grating of the treatment head based on the offset. Exemplarily, based on different correspondence between offsets and motion parameters, the computer device may obtain a motion parameter of the grating, based on the offset between the first target volume and the second target volume and the correspondence, and control the movement of the grating based on the motion parameters. The motion parameters of the grating can be configured to indicate the motion of blades of the grating. In other words, the motion parameters of the grating can be configured to indicate which blades are opened in the grating and at what positions the blades are opened.

In the aforementioned embodiment, since the first target volume in the planning image and the second target volume in the tracking image can be determined, and the offset between the first target volume and the second target volume can be determined, the movement of the grating of the treatment head can be efficiently controlled based on the offset, thereby improving the tracking efficiency during radiotherapy.

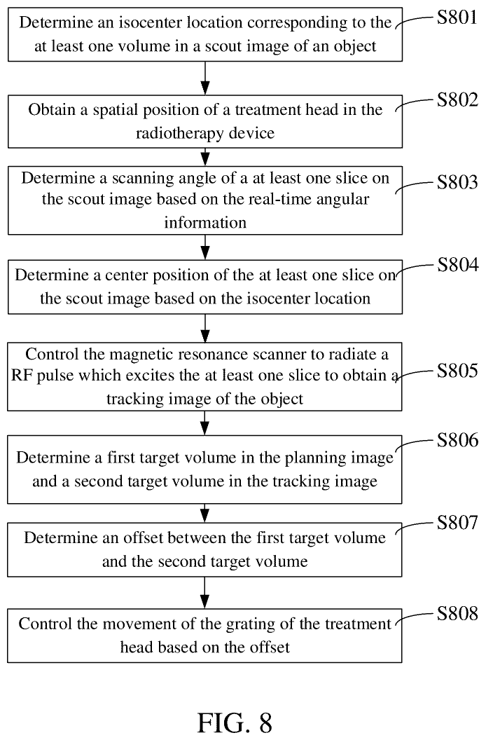

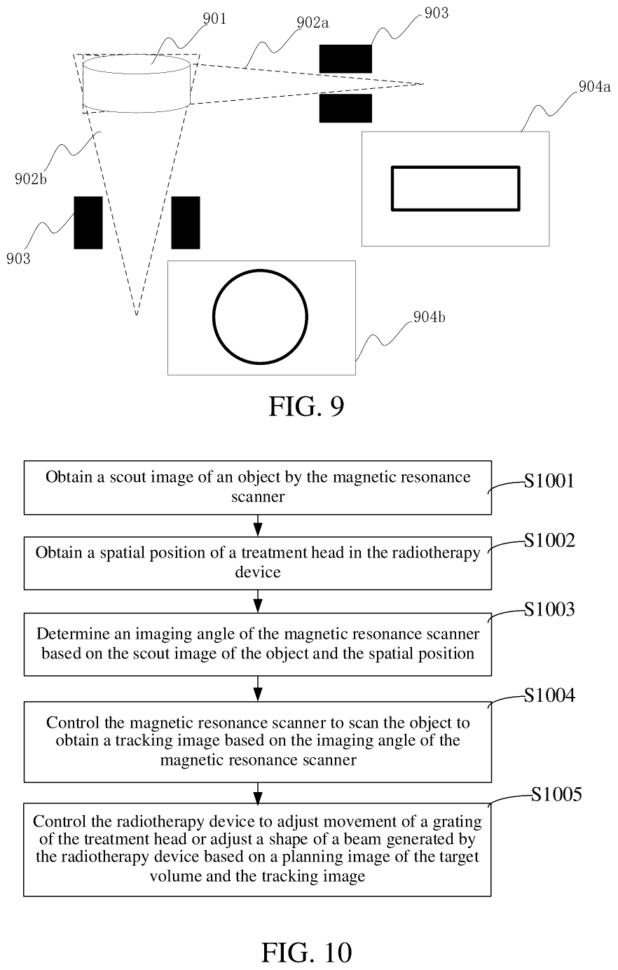

In order to more clearly introduce the target volume tracking method of the present disclosure, it is described here with reference to FIG. 8 and FIG. 9. FIG. 8 is a flow diagram illustrating a target volume tracking method in an embodiment. The computer device may implement the method according to the following steps.

In step S801, an isocenter location corresponding to the target volume in a scout image of an object is determined.

In step S802, a spatial position of a treatment head in the radiotherapy device is obtained. The spatial position includes the real-time angular information of the treatment head attached to a rotatable gantry of the radiotherapy device.

In step S803, a scanning angle of the at least one slice on the scout image is determined based on the real-time angular information.

In step S804, a center position of the at least one slice on the scout image is determined based on the isocenter location.

In step S805, the magnetic resonance scanner is controlled to radiate a RF pulse which excites the at least one slice to obtain a tracking image of the object. The tracking image includes the target volume, and the at least one slice can be determined based on the projection surface of the beam in the treatment head. An angle between the at least one slice and the beam direction of the treatment head is a predetermined value, which may be 90°.

In step S806, a first target volume in the planning image and a second target volume in the tracking image are determined.

In step S807, an offset between the first target volume and the second target volume is determined.

In step S808, the movement of the grating of the treatment head is controlled based on the offset.

Processes of steps S801 to S808 may be implemented with reference to the aforementioned embodiment, and details thereof are not repeated herein. In this way, MRI multi-angle real-time imaging technology can be used to obtain vertical cross-sectional images of the beam to accurately track the maximum movement direction of the present beam. While ensuring tracking accuracy, it can reduce the number of required imaging encoding steps, increase imaging speed, reduce imaging pressure on MRI device, and improve machine stability. Compared with 2D orthogonal surface alternating real-time imaging technology and 3D real-time volumetric imaging technology, it is still a low-slice two-dimensional imaging, which is beneficial to improving the spatial resolution and temporal resolution of the image. In addition, it can reduce computational load in image processing and trajectory coordinate calculations, improve the real-time performance of the algorithm, to achieve a low-latency, high-accuracy target volume tracking capability.

FIG. 9 is a schematic diagram illustrating a target volume tracking method in an embodiment. As shown in FIG. 9, under different beams conditions, acquisition of a certain at least one slice can be performed for the target volume. For example, under the action of a beam limiting device 903, on a condition that the treatment head emits a 90° beam as shown in 902a, the tracking image of the target volume 901 is shown in 904a. On a condition that the treatment head emits a 180° beam as shown in 902b, the tracking image of the target volume 901 is shown in 904b. It can be seen that based on the tracking image, two adaptations can be performed on radiotherapy settings. On the one hand, adjusting the corresponding beam shape in the target volume 901 can achieve conformity with the tumor morphology. On the other hand, adjusting the movement of the multi-leaf collimator in beam limiting device 903, such as adjusting the spacing between adjacent leaves, can achieve follow-up adjustment of the multi-leaf collimator as the tumor moves. Therefore, according to the target volume tracking method provided in this embodiment, the motion state of the target volume can be better obtained, so as to perform radiotherapy methods such as monitoring, gating, and tracking.

FIG. 10 is a flow diagram illustrating a target volume tracking method in another embodiment. In an exemplary embodiment, as shown in FIG. 10, a target volume tracking method is provided. The method is applied to the computer device in FIG. 2, taking that the radiation therapy system is a magnetic resonance guided radiotherapy system, and the medical imaging device is a magnetic resonance scanner as an example, the method includes steps S1001 to S1005.

In step S1001, a scout image of an object is obtained by the magnetic resonance scanner.

In this embodiment, the magnetic resonance scanner scans the object to obtain the scout image of the object firstly. The scout image of the object includes the target volume.

In step S1002, a spatial position of a treatment head in the radiotherapy device is obtained.

Step S1002 may be implemented with reference to step S302, and details thereof are not repeated herein.

In step S1003, an imaging angle of the magnetic resonance scanner is determined based on the scout image of the object and the spatial position.

In this embodiment, after the computer device obtains the scout image of the object and the spatial position of the treatment head, it can determine the imaging angle of the magnetic resonance scanner based on the scout image and the spatial position. It can be understood that the at least one slice of the magnetic resonance scanner can be determined once the imaging angle is determined.

Optionally, the computer device can determine, based on the scout image of the object and the spatial position, an imaging angle that enables the target volume to always be within an irradiation range of the beam and be perpendicular to the beam direction of the treatment head.

In step S1004, the magnetic resonance scanner is controlled to scan the object to obtain a tracking image based on the imaging angle of the magnetic resonance scanner. The tracking image includes a target volume.

In this embodiment, the imaging angle can be determined based on the excitation angle of the radio frequency pulse and the emission timing of the gradient pulse. Furthermore, the computer device can control the excitation angle of the radio frequency pulse and the emission timing of the gradient pulse to control the magnetic resonance scanner to scan the object based on the imaging angle, thereby obtaining a tracking image including the target volume.

In step S1005, the radiotherapy device is controlled to adjust movement of a grating of the treatment head or adjust a shape of a beam generated by the radiotherapy device based on a planning image of the target volume and the tracking image.

In this embodiment, controlling the radiotherapy device to adjust the movement of the grating of the treatment head based on the planning image of the target volume and the tracking image can be implemented with reference the aforementioned embodiment, and details thereof are not repeated herein.

In some embodiments, similarly, the computer device may adjust the shape of the beam generated by the radiotherapy device based on the offset. Optionally, the computer device can adjust a collimator of the radiotherapy device based on the planning image of the target volume and the tracking image to adjust the shape of the beam generated by the radiotherapy device.

In the aforementioned embodiment, since the scout image of the object and the spatial position of the treatment head in the radiotherapy device can be obtained by the magnetic resonance scanner, and then the imaging angle of the magnetic resonance scanner can be determined based on the scout image of the object and the spatial position, the imaging angle is not fixed, nor does it change alternately in only three directions, but it can be determined based on the scout image and the spatial position of the treatment head in the radiotherapy device. Therefore, the imaging angle can be adaptively changed based on the beam of the treatment head, which is more accurate. Furthermore, based on the imaging angle of the magnetic resonance scanner, the magnetic resonance scanner can be controlled to scan the object to obtain a target volume tracking image, thereby improving the accuracy of target volume tracking. Furthermore, based on the planning image of the target volume and the tracking image, the accuracy of radiotherapy can be improved by controlling the radiotherapy device to adjust the movement of the grating of the treatment head or the shape of the beam generated by the radiotherapy device.

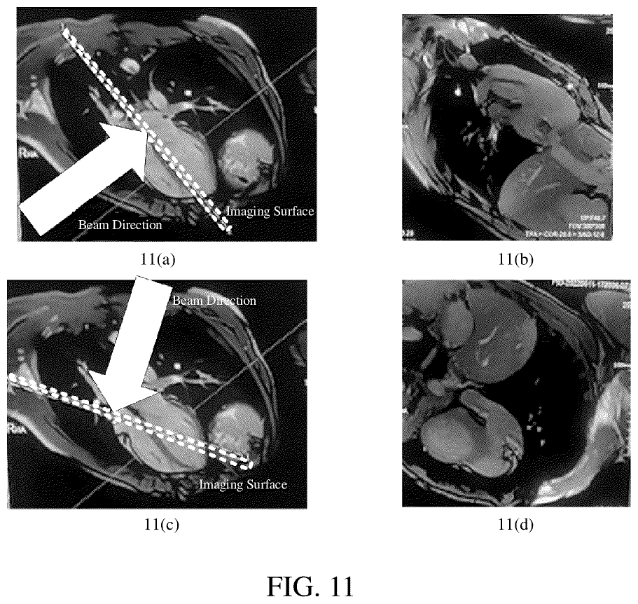

FIG. 11 is a schematic diagram illustrating the effect of target volume tracking in an embodiment. FIG. 11 shows tracking images at two imaging angles. White arrows in 11 (a) and 11 (c) refer to the beam directions of the treatment head, i.e., beam angles. White dotted lines in 11 (a) and 11 (c) refer to the imaging plane of the magnetic resonance scanner, i.e., the at least one slice.

Referring to 11 (a) and 11 (b). Under the beam direction shown in 11 (a), the magnetic resonance scanner may scan the object based on the imaging plane shown in 11 (a). The tracking image obtained by the scanning is shown in 11 (b).

Referring to 11 (c) and 11 (d). Under the beam direction shown in 11 (c), the magnetic resonance scanner may scan the object based on the imaging plane shown in 11 (c), and the tracking image obtained by the scanning is shown in 11 (d).

The target volume tracking method applied to the magnetic resonance guided radiotherapy system is described above. It can be understood that the target volume tracking method provided in the present disclosure can further be applied to other types of guided radiotherapy systems, which will be described in detail below.

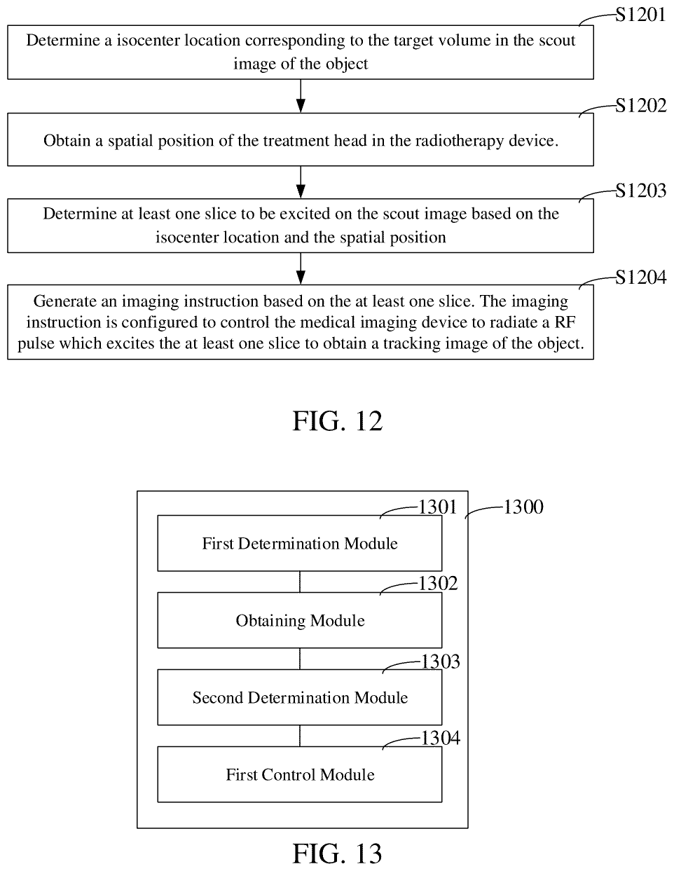

FIG. 12 is a flow diagram illustrating a target volume tracking method in another embodiment. In an exemplary embodiment, as shown in FIG. 12, a target volume tracking method is provided, which is illustrated by applying the method to the computer device in FIG. 2, including the following steps S1201 to S1204.

In step S1201, an isocenter location corresponding to the target volume in the scout image of the object is determined.

In this embodiment, the computer device can directly obtain the isocenter location corresponding to the target volume in the scout image of the object, or obtain the scout image of the object by the medical imaging device to further determine the isocenter location corresponding to the target volume in the scout image. Specifically, the medical imaging device may include, but is not limited to, CT device, ultrasound device, and other imaging device that can obtain an anatomical structure of the object. Furthermore, the computer device can obtain a scout image of an object by scanning the object through the medical imaging device, and the scout image can illustrate the anatomical structure of the object. Furthermore, the scout image includes the target volume. The scout image can illustrate the anatomical structure of the target volume. The computer device determines the isocenter location corresponding to the target volume in the scout image.

In step S1202, a spatial position of the treatment head in the radiotherapy device is obtained. The radiotherapy device is integrated with a medical imaging device.

In step S1203, at least one slice to be excited on the scout image is determined based on the isocenter location and the spatial position.

In this embodiment, step S1202 may be implemented with reference to step S302, and step S1203 may be implemented with reference to step S303, and details thereof are not repeated herein.

In step S1204, an imaging instruction is generated based on the at least one slice. The imaging instruction is configured to control the medical imaging device to radiate a RF pulse which excites the at least one slice to obtain a tracking image of the object. The tracking image includes the target volume.

In this embodiment, after the computer device determines the position of the at least one slice, the computer device can generate imaging instructions for controlling the medical imaging device to control the medical imaging device to scan the object based on the at least one slice, thereby obtaining a tracking image including the target volume. Taking the medical imaging device as a CT device as an example, the computer device can send an imaging instruction including the at least one slice to the CT device. After receiving the imaging instruction, the CT device can scan the object based on the at least one slice to obtain a tracking image.

In the aforementioned embodiment, the isocenter location corresponding to the target volume in the scout image of the object can be obtained, and the spatial position of the treatment head in the radiotherapy device can be obtained. Since the radiotherapy device and the medical imaging device are integrated, and the scout image is obtained by scanning the medical imaging device and can reflect the anatomical structure of the object, the at least one slice can be determined on the scout image based on the isocenter location and spatial position. In this way, the at least one slice is not fixed, nor does it change alternately only in three directions, but can be determined based on the spatial position of the treatment head in the radiotherapy device. Therefore, the at least one slice can be adaptively changed based on the beam of the treatment head. Furthermore, since the imaging instruction is configured to control a medical imaging device to radiate a RF pulse which excites the at least one slice to obtain the tracking image of the object and the tracking image includes the target volume, after generating the imaging instructions for controlling the medical imaging device based on the at least one slice, a more accurate tracking image including the target volume can be obtained, thereby improving the accuracy of target volume tracking.

In an embodiment, optionally, the target volume tracking method further includes the following steps.

A control instruction is generated based on a planning image of the target volume and the tracking image. The control instruction is configured to control movement of a grating of the treatment head or control a shape of a beam generated by the radiotherapy device.

In this embodiment, the computer device can obtain the planning image of the target volume. For example, the medical imaging device may scan the target volume before surgery and send the planning image obtained after the scanning to the computer device.

Furthermore, a control instruction for the radiotherapy device can be generated based on the planning image of the target volume and the tracking image. Exemplarily, when the treatment head moves to a spatial position A, the computer device can generate a control instruction A based on the planning image and the tracking image A. When the treatment head moves to a spatial position B, the computer device can generate a control instruction B based on the planning image and the tracking image B.

Optionally, the computer device may determine an offset between the first target volume and the second target volume based on the first target volume in the planning image and the second target volume in the tracking image, and generate a control instruction for the radiotherapy device based on the offset. The computer device can further determine a target beam shape based on a difference between the planning image and the tracking image, and generate a control instruction for the radiotherapy device based on the target beam shape.

Furthermore, the computer device can control the movement of the grating of the treatment head or the shape of the beam generated by the radiotherapy device through the control instruction. For example, the computer device sends a control instruction to the radiotherapy device to control the movement of the grating of the treatment head or the shape of the beam generated by the radiotherapy device according to the control instruction.

In the aforementioned embodiment, since the control instruction for the radiotherapy device can be generated based on the planning image of the target volume and the tracking image to control the movement of the grating of the treatment head or the shape of the beam generated by the radiotherapy device, so that the tracking accuracy during the radiotherapy is improved.

It should be understood that, although the steps in the flowcharts involved in the embodiments described above are displayed sequentially as indicated by the arrows, these steps are not necessarily executed sequentially in the order indicated by the arrows. Unless otherwise specified herein, there is no strict order limitation for the execution of these steps, and these steps may be executed in other orders. Moreover, at least a part of the steps in the flowcharts involved in the embodiments described above may include multiple steps or multiple stages. These steps or stages are not necessarily executed at the same time, but can be executed at different times. The execution order of these steps or stages is not necessarily sequential, but can be executed in turn or alternately with other steps or at least part of the steps or stages in other steps.

Based on the same inventive concept, an embodiment of the present disclosure also provides a target volume tracking apparatus for implementing the target volume tracking method described above. The solution to the problem provided by the apparatus is similar to the solution described in the above method, so the specific limitations in one or more target volume tracking apparatus embodiments provided below can be implemented with reference to the limitations on the target volume tracking method above, and details thereof are not repeated herein.

FIG. 13 is a block diagram illustrating a configuration of a target volume tracking apparatus in an embodiment. In an exemplary embodiment, as shown in FIG. 13, a target volume tracking apparatus 1300 is provided, which is applied to a magnetic resonance guided radiotherapy system. The magnetic resonance guided radiotherapy system includes a magnetic resonance scanner and a radiotherapy device. The target volume tracking apparatus 1300 includes: a first determination module 1301, an obtaining module 1302, a second determination module 1303 and a first control module 1304.

The first determination module 1301 is configured to determine an isocenter location corresponding to the target volume in a scout image of an object.

The obtaining module 1302 is configured to obtain a spatial position of a treatment head in the radiotherapy device.

The second determination module 1303 is configured to determine at least one slice to be excited on the scout image based on the isocenter location and the spatial position.

The first control module 1304 is configured to control the magnetic resonance scanner to radiate a RF pulse which excites the at least one slice to obtain a tracking image of the object. The tracking image includes the target volume.

In the above-mentioned target volume tracking apparatus, since the isocenter location corresponding to the target volume in the scout image of the object can be obtained, and the spatial position of the treatment head in the radiotherapy device can be obtained, the at least one slice can be determined on the scout image based on the isocenter location and the spatial position. In this way, the at least one slice is not fixed, nor does it change alternately only in three directions. Instead, it can be determined based on the spatial position of the treatment head in the radiotherapy device. Therefore, the at least one slice can be adaptively changed based on the beam of the treatment head. Furthermore, based on the at least one slice, the magnetic resonance scanner is controlled to scan the object to obtain the tracking image, so that a more accurate tracking image including the target volume can be obtained, thereby improving the accuracy of target volume tracking.

Optionally, the at least one slice of the object is affected by respiratory movement or cardiac movement.

Optionally, the spatial position includes real-time angular information of the treatment head relative to the central point in the radiotherapy device. The at least one slice includes a scanning angle and a central position of the at least one slice on the scout image. The second determination module 1303 includes a first determining unit and a second determining unit.

The first determining unit is configured to determine the scanning angle based on the real-time angular information.

The second determining unit is configured to determine the center position based on the isocenter location.

Optionally, the second determining module 1303 includes a third determining unit, a fourth determining unit, and a fifth determination unit.

The third determining unit is configured to determine a line connecting the isocenter location and the spatial position.

The fourth determining unit is configured to determine a circular section where the line and a gantry of the treatment head are located.

The fifth determination unit is configured to determine the at least one slice parallel to the circular section on the scout image based on the isocenter location.

Optionally, the target volume tracking apparatus 1300 further includes a second control module.

The second control module is configured to control the movement of a grating of the treatment head based on a planning image of the target volume and the tracking image.

Optionally, the second control module includes a sixth determination unit, a seventh determination unit, and a control unit.

The sixth determination unit is configured to determine a first target volume in the planning image and a second target volume in the tracking image.

The seventh determination unit is configured to determine an offset between the first target volume and the second target volume.

The control unit is configured to control the movement of a grating of the treatment head based on the offset.

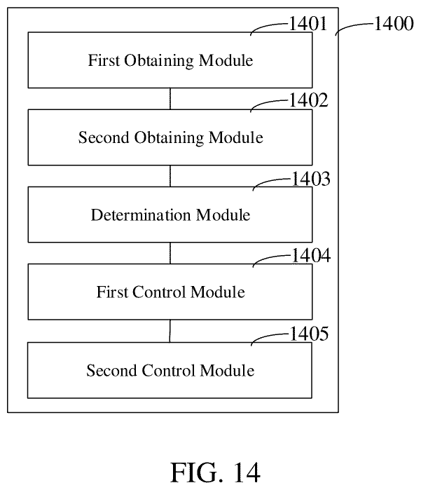

FIG. 14 is a block diagram illustrating a configuration of a target volume tracking apparatus in another embodiment. In an exemplary embodiment, as shown in FIG. 14, a target volume tracking apparatus 1400 is provided, which is applied to a magnetic resonance guided radiotherapy system. The magnetic resonance guided radiotherapy system includes a magnetic resonance scanner and a radiotherapy device. The target volume tracking apparatus 1400 includes: a first obtaining module 1401, a second obtaining module 1402, a determination module 1403, a first control module 1404 and a second control module 1405.

The first obtaining module 1401 is configured to obtain a scout image of an object by the magnetic resonance scanner.

The second obtaining module 1402 is configured to obtain a spatial position of a treatment head in the radiotherapy device.

The determination module 1403 is configured to determine an imaging angle of the magnetic resonance scanner based on the scout image of the object and the spatial position.

The first control module 1404 is configured to control the magnetic resonance scanner to scan the object to obtain a tracking image based on the imaging angle of the magnetic resonance scanner. The tracking image includes a target volume.

The second control module 1405 is configured to control the radiotherapy device to adjust the movement of a grating of the treatment head or adjust a shape of a beam generated by the radiotherapy device based on a planning image of the target volume and the tracking image.

In the aforementioned embodiment, since the scout image of the object and the spatial position of the treatment head in the radiotherapy device can be obtained by the magnetic resonance scanner, and then the imaging angle of the magnetic resonance scanner can be determined based on the scout image of the object and the spatial position, the imaging angle is not fixed, nor does it change alternately in only three directions, but it can be determined based on the scout image and the spatial position of the treatment head in the radiotherapy device. Therefore, the imaging angle can be adaptively changed based on the beam of the treatment head, which is more accurate. Furthermore, based on the imaging angle of the magnetic resonance scanner, the magnetic resonance scanner can be controlled to scan the object to obtain a target volume tracking image, thereby improving the accuracy of target volume tracking. Furthermore, based on the planning image of the target volume and the tracking image, the accuracy of radiotherapy can be improved by controlling the radiotherapy device to adjust the movement of the grating of the treatment head or the shape of the beam generated by the radiotherapy device.

Each module in the above-mentioned target volume tracking apparatus can be fully or partially implemented by software, hardware or a combination thereof. The above modules may be embedded in or independent of a processor in a computer device in the form of hardware, or may be stored in a memory in a computer device in the form of software, so that the processor can call and execute operations corresponding to the above modules.

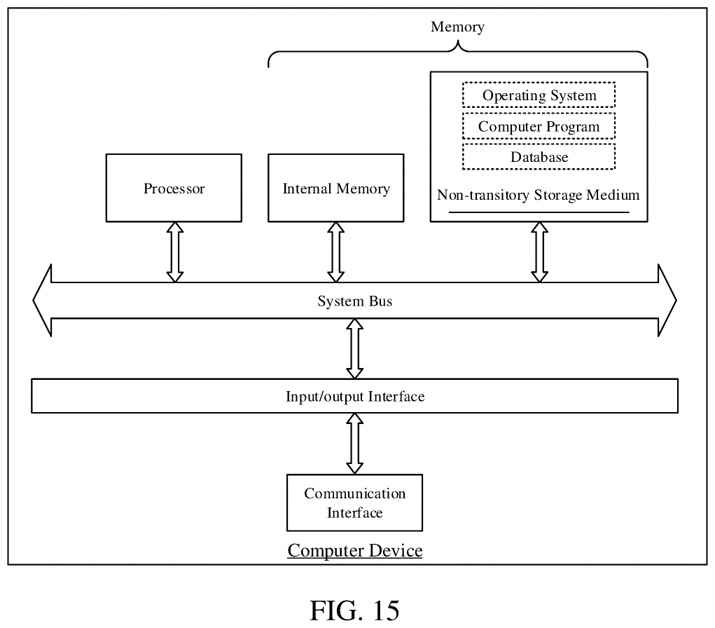

FIG. 15 is a diagram illustrating an internal configuration of a computer device in an embodiment. In an exemplary embodiment, a computer device is provided. The computer device may be a server, and its internal configuration diagram may be as shown in FIG. 15. The computer device includes a processor, a memory, an input/output interface (I/O) and a communication interface. The processor, the memory and the input/output interface are connected via a system bus, and the communication interface is connected to the system bus via the input/output interface, the processor of the computer device is configured to provide computing and control capabilities. The memory of the computer device includes a non-transitory storage medium and an internal memory. The non-transitory storage medium stores an operating system, a computer program and a database. The internal memory provides an environment for the operation of the operating system and computer programs in the non-transitory storage medium. The database of the computer device is configured to store relevant data. The input/output interface of the computer device is configured to exchange information between the processor and external devices. The communication interface of the computer device is configured to communicate with an external terminal via a network connection. When the computer program is executed by a processor, a target volume tracking method is implemented.

Those skilled in the art will understand that the configuration shown in FIG. 15 is merely a block diagram illustrating a partial configuration related to the scheme of the present disclosure, and does not constitute a limitation on the computer device to which the scheme of the present disclosure is applied. The specific computer device may include more or fewer components than shown in the figure, or combine certain components, or have a different arrangement of components.

In an exemplary embodiment, a computer device is provided, including a memory and a processor. A computer program is stored in the memory, and the computer program, when executed by the processor, causes the processor to implement the following steps: determining an isocenter location corresponding to the target volume in a scout image of an object; obtaining a spatial position of a treatment head in the radiotherapy device, the radiotherapy device being integrated with the medical imaging device; determining at least one slice to be excited on the scout image based on the isocenter location and the spatial position; and generating an imaging instruction based on the at least one slice. The imaging instruction is configured to control the medical imaging device to radiate a RF pulse which excites the at least one slice to obtain a tracking image of the object. The tracking image includes the target volume.

In an embodiment, the computer program, when executed by the processor, causes the processor to further implement the following step: generating a control instruction based on a planning image of the target volume and the tracking image. The control instruction is configured to control movement of a grating of the treatment head or control a shape of a beam generated by the radiotherapy device.

In an embodiment, a computer-readable storage medium is provided, on which a computer program is stored. When the computer program is executed by a processor, the steps in the above-mentioned method embodiments are implemented.

In an embodiment, a computer program product is provided, including a computer program. When the computer program is executed by a processor, the steps in the above-mentioned method embodiments are implemented.

A person skilled in the art can understand that all or part of the processes in the above-mentioned embodiment methods can be implemented by instructing related hardware through a computer program. The computer program can be stored in a non-transitory computer-readable storage medium. When the computer program is executed, it can include the processes of the embodiments of the above-mentioned methods. Any reference to a memory, a database, or other medium used in the embodiments provided in the present disclosure may include at least one of a non-transitory memory and a transitory memory. Non-transitory memory may include read-only memory (ROM), magnetic tape, floppy disk, flash memory, optical storage, high-density embedded non-transitory memory, resistive random access memory (ReRAM), magnetoresistive random access memory (MRAM), ferroelectric random access memory (FRAM), phase change memory (PCM), graphene memory, etc. Transitory memory may include random access memory (RAM) or external cache memory, etc. By way of illustration and not limitation, RAM may be in various forms, such as Static Random Access Memory (SRAM) or Dynamic Random Access Memory (DRAM). The database involved in each embodiment provided in the present disclosure may include at least one of a relational database and a non-relational database. Non-relational databases may include, but are not limited to, distributed databases based on blockchain. The processor involved in each embodiment provided in the present disclosure may be a general-purpose processor, a central processing unit, a graphics processor, a digital signal processor, a programmable logic device, a data processing logic device based on quantum computing, an artificial intelligence (AI) processor, etc., but is not limited thereto.

The technical features of the above embodiments can be randomly combined. To simplify the description, not all possible combinations of the technical features in the above embodiments are described. However, as long as there is no contradiction in the combination of these technical features, all the combinations should be considered to be included within the scope of this specification.

The above-described embodiments only illustrate several embodiments of the present disclosure, and the descriptions of which are relatively specific and detailed, but should not be construed as limiting the scope of the patent disclosure. It should be noted that, for those of ordinary skill in the art, several modifications and improvements can be made without departing from the concept of the present disclosure, and these all fall within the protection scope of the present disclosure. Therefore, the protection scope of the present disclosure should be determined by the appended claims.

What is claimed is:

1. A target volume tracking method applied to a magnetic resonance guided radiotherapy system, the magnetic resonance guided radiotherapy system comprising a magnetic resonance scanner and a radiotherapy device, the method comprising:
determining an isocenter location corresponding to a target volume in a scout image of an object;
obtaining a spatial position of a treatment head in the radiotherapy device;
determining at least one slice to be excited on the scout image based on the isocenter location and the spatial position; and
controlling the magnetic resonance scanner to radiate a RF pulse which excites the at least one slice to obtain a tracking image of the object, the tracking image comprising the target volume.

2. The method according to claim 1, wherein determining the isocenter location corresponding to the target volume in the scout image of the object comprises:
obtaining the scout image by the magnetic resonance scanner, and determining the isocenter location in the scout image.

3. The method according to claim 2, wherein determining the isocenter location in the scout image comprises:
determining the isocenter location based on the scout image and a coordinate transformation relationship, the coordinate transformation relationship comprising a transformation relationship between a real space coordinate system and an image coordinate system corresponding to the scout image.

4. The method according to claim 1, wherein the at least one slice of the object is affected by respiratory movement or cardiac movement.

5. The method according to claim 4, wherein the at least one slice is a two-dimensional slice.

6. The method according to claim 4, wherein an angle between the at least one slice and a beam direction of the treatment head is greater than or equal to 80°, and less than or equal to 110°.

7. The method according to claim 6, wherein the angle between the at least one slice and the beam direction of the treatment head is 90°.

8. The method according to claim 1, wherein the spatial position comprises real-time angular information of the treatment head attached to a rotatable gantry of the radiotherapy device, and determining the at least one slice on the scout image based on the isocenter location and the spatial position comprises:
determining a scanning angle of the at least one slice on the scout image based on the real-time angular information; and
identifying a median axis of the target volume based on the isocenter location.

9. The method according to claim 1, wherein determining the at least one slice on the scout image based on the isocenter location and the spatial position comprises:
determining a line connecting the isocenter location and the spatial position;
determining a circular section where the line and a gantry of the treatment head are located; and
determining, based on the isocenter location, the at least one slice parallel to the circular section on the scout image.

10. The method according to claim 1, wherein the method further comprises:
controlling movement of a grating of the treatment head based on a planning image of the target volume and the tracking image.

11. The method according to claim 10, wherein controlling the movement of a grating of the treatment head based on the planning image of the target volume and the tracking image comprises:
determining a first target volume in the planning image and a second target volume in the tracking image;
determining an offset between the first target volume and the second target volume; and
controlling the movement of the grating of the treatment head based on the offset.

12. A target volume tracking method applied to a magnetic resonance guided radiotherapy system, the magnetic resonance guided radiotherapy system comprising a magnetic resonance scanner and a radiotherapy device, the method comprising:
obtaining a scout image of an object by the magnetic resonance scanner;
obtaining a spatial position of a treatment head in the radiotherapy device;
determining an imaging angle of the magnetic resonance scanner based on the scout image of the object and the spatial position;
controlling, based on the imaging angle of the magnetic resonance scanner, the magnetic resonance scanner to scan the object to obtain a tracking image, the tracking image comprising the target volume; and
controlling, based on a planning image of the target volume and the tracking image, the radiotherapy device to adjust movement of a grating of the treatment head or adjust a shape of a beam generated by the radiotherapy device.

13. A computer device comprising a memory and a processor, the memory storing a computer program, wherein the computer program, when executed by the processor, causes the processor to implement:
determining an isocenter location corresponding to a target volume in a scout image of an object;
obtaining a spatial position of a treatment head in a radiotherapy device;
determining at least one slice to be excited on the scout image based on the isocenter location and the spatial position; and
generating an imaging instruction based on the at least one slice, the imaging instruction being configured to control a medical imaging device to radiate a RF pulse which excites the at least one slice to obtain a tracking image of the object, and the tracking image comprising the target volume.

14. The computer device according to claim 13, wherein the computer program, when executed by the processor, causes the processor to further implement:
obtaining the scout image by the medical imaging device, and determining the isocenter location in the scout image.

15. The computer device according to claim 14, wherein the computer program, when executed by the processor, causes the processor to further implement:
determining the isocenter location based on the scout image and a coordinate transformation relationship, the coordinate transformation relationship comprising a transformation relationship between a real space coordinate system and an image coordinate system corresponding to the scout image.

16. The computer device according to claim 13, wherein the computer program, when executed by the processor, causes the processor to further implement:
generating a control instruction based on a planning image of the target volume and the tracking image, the control instruction being configured to control movement of a grating of the treatment head.

17. The computer device according to claim 13, wherein the computer program, when executed by the processor, causes the processor to further implement:
generating a control instruction based on a planning image of the target volume and the tracking image, the control instruction being configured to control a shape of a beam generated by the radiotherapy device.

18. The computer device according to claim 13, wherein the computer program, when executed by the processor, causes the processor to further implement:
determining a scanning angle of the at least one slice on the scout image based on real-time angular information of the treatment head attached to a rotatable gantry of the radiotherapy device, the spatial position comprising the real-time angular information; and
determining a center position of the at least one slice on the scout image based on the isocenter location.

19. The computer device according to claim 13, wherein the computer program, when executed by the processor, causes the processor to further implement:
determining a line connecting the isocenter location and the spatial position;
determining a circular section where the line and a gantry of the treatment head are located; and
determining, based on the isocenter location, the at least one slice parallel to the circular section on the scout image.

20. The computer device according to claim 13, wherein the computer program, when executed by the processor, causes the processor to further implement:
controlling movement of a grating of the treatment head based on a planning image of the target volume and the tracking image.
